(19) United States
(12) Patent Application Publication
(10) Pub. No.: US 2026/0115705 A1
(43) Pub. Date: Apr. 30, 2026

(54) LOW-ODOR PHOSPHAZENE CATALYST AND USE THEREOF

(71) Applicant: CHANGHUA CHEMICAL TECHNOLOGY CO., LTD., Jiangsu (CN)

(72) Inventors: Junfei ZHAO, Jiangsu (CN); Jia GU, Jiangsu (CN); Peng LI, Jiangsu (CN); Qiang BIAN, Jiangsu (CN)

(73) Assignee: CHANGHUA CHEMICAL TECHNOLOGY CO., LTD., Jiangsu (CN)

(21) Appl. No.: 19/082,084

(22) Filed: Mar. 17, 2025

Related U.S. Application Data
(63) Continuation of application No. PCT/CN2024/129866, filed on Nov. 5, 2024.

(30) Foreign Application Priority Data
Oct. 31, 2024 (CN) 202411537427.5

(51) Int. Cl. B01J 31/02; B01J 37/00; B01J 37/08; C08G 65/00; C08G 71/04

(52) U.S. Cl. CPC B01J 31/0265; B01J 37/0045; B01J 37/009; B01J 37/08; C08G 65/00; C08G 71/04

(57) ABSTRACT

A continuous preparation method for a low-odor phosphazene catalyst and use thereof are provided. The phosphazene catalyst includes a phosphazene cation and an alkali metal salt compound anion represented by General Formula (I); and the phosphazene catalyst is prepared by using phosphorus pentahalide and a guanidine compound as raw materials, dissolving the phosphorus pentahalide in an organic non-benzene solvent to react under heating conditions, then obtaining an organic phosphonium salt solution containing the organic non-benzene solvent and a halogenated phosphorus compound, adding the alkali metal salt compound to react under an inert atmosphere and heating conditions, and then filtering, distilling under a reduced pressured, purifying and vacuum drying. The phosphazene catalyst is used in the preparation of polyether polyols and polyurethane foam materials.

### CROSS-REFERENCE TO RELATED APPLICATION

This application is a continuation of international application of PCT application serial no. PCT/CN2024/129866, filed on Nov. 5, 2024, which claims the priority benefit of China application no. 202411537427.5, filed on Oct. 31, 2024. The entirety of each of the above-mentioned patent applications is hereby incorporated by reference herein and made a part of this specification.

### TECHNICAL FIELD

The present disclosure relates to catalysts and use thereof, and particularly relates to a low-odor phosphazene catalyst and use thereof.

### BACKGROUND

Having excellent ring-opening catalytic ability, high activity and stability in a reaction process, phosphazene catalysts produce few by-products, and can be used for preparing polyether polyols with higher molecular weights and higher activity, while maintaining low unsaturation and few by-products. Some patents describe the processes for preparing the phosphazene catalysts, which involves complicated technology that is time-consuming and inefficient. For example, patents DE102006010034 and CN102171272B both involve multiple reactions and processing steps to obtain the phosphazene catalysts, the processes thereof involve a variety of raw materials, solvents, and water, which are time-consuming and energy-consuming, generate wastewater, and lead to waste pollution. Amid rapid economic development at present, energy waste and environmental pollution are becoming increasingly serious, which forces people to pay greater attention to energy conservation and efficient improvement in industrial production, and to give top priority to the environmental protection at all times.

In addition, the prior art mentions that the phosphazene catalyst process can be used to prepare polyether polyols with high molecular weight and low unsaturation. However, the polyether polyols obtained through a phosphazene catalytic system have a strong odor, making subsequent treatment more complex. The odor thereof mainly arises from two sources: one is an impact of the selected phosphazene catalyst, and the other is by-products and impurities, such as small molecular alcohols, aldehydes, ketones, and benzene, generated from a reaction process, and the impurities often affect the performance of polyurethane materials prepared from the polyether polyols. Currently, most of the domestic and overseas polyether manufacturers adopt post-treatment methods of crude polyether, such as neutralization and adsorption, which optimize both process and raw materials, but the odor thereof has not been significantly improved. With the continuous progress of society and people's constant pursuit of a better and healthier life, customer demands for the odor of polyether polyols and products made from the same are continuously increasing, these factors force researchers to optimize not only the odor of polyether polyols but also a catalyst end, so as to reduce the odor of polyethers and minimize harmful substances arising therefrom.

The prior art discloses a catalyst for production of polyalkylene glycol, which uses phosphorus pentachloride and tetramethylguanidine to prepare a phosphazene-halonium salt intermediate in a benzene solvent. After purification, an anion is replaced with a hydroxide, and is then mixed with an active hydrogen compound and heated to obtain a catalyst for the manufacture of polyalkylene glycol. The catalyst is used to further catalyze the polymerization of epoxides such as propylene oxide, and is applied in the field of polyurethane foam. However, the catalyst produced by using toluene as a solvent has many problems, such as strong odor and excessive benzene series. The prior art also discloses an organic alcohol salt phosphazene catalyst, which is prepared by synthesizing an intermediate in a benzene solvent using phosphorus trichloride and tetramethylguanidine, followed by ion exchange. The catalyst in the present disclosure can also be used in the ring-opening polymerization of epoxy compound to prepare polyether polyols with high molecular weight and low unsaturation.

In summary, the preparation of phosphazene catalysts in the prior art usually requires various different organic solvents containing benzene series. After the reaction, the organic solvents used therein need to be extracted and washed with water, and the reaction products can be obtained only through ion exchange, which will generate a large amount of wastewater and waste solvents. Therefore, the phosphazene catalysts in the prior art generally have many problems, such as toxic and hazardous raw materials or solvents in the manufacture process, and involving numerous steps, complicated operations, low economic efficiency, and strong odor of the prepared polyether products and polyurethane foam, excessive benzene series, and the like.

### SUMMARY

A first objective of the present disclosure is to provide a low-odor phosphazene catalyst with a simple preparation process.

A second objective of the present disclosure is to provide use of the low-odor phosphazene catalyst in the preparation of polyether polyols and polyurethane foam materials.

Technical solution: the low-odor phosphazene catalyst of the present disclosure includes a phosphazene cation and an alkali metal salt compound anion represented by General Formula (I);

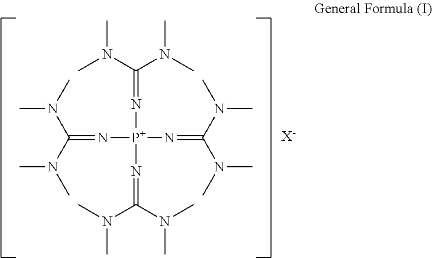

in the above General Formula (I), X represents an alkali metal salt compound, and X-represents an alkali metal anion formed by the departure of a metal ion from X, where X is selected from one of sodium alkoxide, potassium alkoxide, sodium carboxylate, potassium carboxylate, sodium dihydrogen phosphate, or potassium dihydrogen phosphate; and

a continuous preparation method for the low-odor phosphazene catalyst includes the following steps:

(1) dissolving phosphorus pentahalide in an organic solvent to obtain an organic phosphorus pentahalide solution; adding dropwise a solution of guanidine compound to the organic phosphorus pentahalide solution under an inert atmosphere and at a temperature of −15° C.-5° C.; bringing a temperature of the solution to room temperature after the addition is completed, stirring at room temperature, then heating in an oil bath to facilitate continuous reaction, cooling to room temperature upon completion of the reaction, filtering to remove a precipitate to obtain an organic phosphonium salt solution containing the organic solvent and a halogenated phosphorus compound;

(2) adding the alkali metal salt compound to the organic phosphonium salt solution obtained in the step (1); performing the reaction under an inert atmosphere and heating conditions, cooling and filtering after the completion of reaction to obtain a filtrate, distilling the filtrate under a reduced pressure to obtain a crude product of phosphazene compound; and

the organic solvent is selected from at least one of nitriles or ethers; and the alkali metal salt compound is selected from one of sodium alkoxide, potassium alkoxide, sodium carboxylate, potassium carboxylate, or phosphate metal salt.

Further, the organic solvent is selected from at least one of propionitrile, butyronitrile, adiponitrile, propyl ether, butyl ether, or 1,4-dioxane; and the alkali metal salt compound is selected from one of potassium methoxide, sodium methoxide, potassium ethoxide, sodium ethoxide, potassium formate, sodium formate, potassium acetate, sodium acetate, potassium dihydrogen phosphate, or sodium dihydrogen phosphate.

In the step (1), the phosphorus pentahalide is selected from phosphorus pentabromide or phosphorus pentachloride; and the guanidine compound is 1,1,3,3-tetramethylguanidine.

In the step (1), a mass ratio of the phosphorus pentahalide to the organic solvent is 1:6-12; a molar ratio of the phosphorus pentahalide to the guanidine compound is 1:5-11; a temperature is 0-5° C.; and a temperature for oil bath heating is 80-120° C.; and an oil bath stirring reaction lasts for 3-8 h.

In the step (2), a molar ratio of the organic phosphonium salt solution to the alkali metal salt compound is 1:1-2; and a reaction temperature is 50-80° C., the reaction lasts for 3-8 h, and a reaction pressure is atmospheric pressure.

The crude product of phosphazene compound obtained in the step (2) is subjected to recrystallization or slurry for purification.

Further, steps for the recrystallization are as follows: the crude product of phosphazene compound is subjected to recrystallization using a purification solvent, then filtering and drying to obtain a white powdery low-odor phosphazene catalyst; where the purification solvent is selected from a mixed solution of at least one of nitriles or alcohols and at least one of ethers or alkanes; and a volume ratio of at least one of nitriles or alcohols to at least one of ethers or alkanes is 1:2-300, and preferably 1:50-100; and

Further, steps for the slurry are as follows: the crude product of phosphazene compound is subjected to slurry using a purification solvent, then filtering and drying to obtain a white powdery low-odor phosphazene catalyst; where the purification solvent is selected from a mixed solution of at least one of nitriles or alcohols and at least one of ethers or alkanes; and a volume ratio of at least one of nitriles or alcohols to at least one of ethers or alkanes is 1:2-500, and preferably 1:100-200.

Use of the low-odor phosphazene catalyst in the preparation of polyether polyols and polyurethane foam materials.

The low-odor phosphazene catalyst represented in General Formula (I) is mixed with an active hydrogen compound Y and subjected to heating treatment to obtain a salt of phosphazene cation and active hydrogen compound anion represented in General Formula (II), which is used to prepare a polyether polyol.

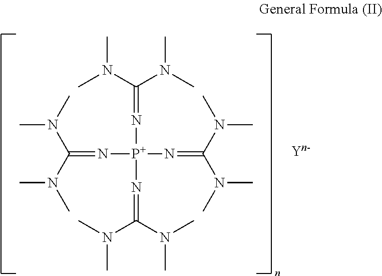

In the above General Formula (II), n is a real number greater than 0 but less than or equal to 8, and Y″-represents an anion of the active hydrogen compound formed by the departure of n protons from the active hydrogen compound Y; and Y is a polyether polyol with a functionality of 2-8 and a molecular weight of 300-2000.

Further, n is a real number greater than 0 but less than or equal to 1, and Y is a polyether polyol with a functionality of 2-6 and a molecular weight of 400-1200.

Beneficial effects: compared with the prior art, the present disclosure has the following advantages:

(1) the phosphazene catalyst of the present disclosure uses only one non-benzene inert solvent that does not participate in the reaction during the preparation, thereby with no need to frequently change the solvent. By using the same solvent, steps of extraction, distillation, and the like, are not required, thereby reducing energy consumption and the generation of wastewater and waste materials during the process. The continuous preparation method for the phosphazene catalyst allows for the efficient production of the phosphazene catalyst without benzene series, which also plays an active role in environmental protection; (2) the catalyst synthesized in this way can be purified by slurry or recrystallization, which can make the phosphazene catalyst have lower odor and higher purity. Most importantly, the preparation process provides strong support for the industrial production of phosphazene catalyst; (3) the purified catalyst has low impurity content, which can significantly inhibit the small molecular impurities in the polyol produced subsequently. In addition, the odor of the prepared polyurethane foam can also be significantly improved; and (4) when the value of n in General Formula (II) is greater than 0 but less than or equal to 1, the proportion of the catalyst in the initiator is small, and the catalyst maintains high catalytic activity during synthesis of polyether, which cannot only reduce the corporate cost, but also enhance energy efficiency, play an active role in social energy conservation and emission reduction.

### DETAILED DESCRIPTIONS OF THE EMBODIMENTS

The present disclosure will be further described in detail below.

Sources of raw materials in various examples of the present disclosure are shown in Table 1 below.

TABLE 1
List of raw materials

| | Brand and | | |
|---|---|---|---|
| Raw materials | specifications | Specification | Supplier |
| Polyether | CHE-204 | Hydroxyl value 280 mgKOH/g | ChangHua Chemical |
| polyol | | Functionality 3 | Technology Co., Ltd. |
| | | Molecular weight 400 g/mol | |
| Polyether | CHE-307 | Hydroxyl value 240 mgKOH/g | ChangHua Chemical |
| polyol | | Functionality 2 | Technology Co., Ltd. |
| | | Molecular weight 700 g/mol | |
| Polyether | NJ-6249 | Hydroxyl value 490 mgKOH/g | Jurong Ningwu New |
| polyol | | Functionality 6 | Material Co., Ltd. |
| | | Molecular weight 650 g/mol | |
| Catalyst | Dabco NE-1091 | / | Huntsman |
| Foam stabilizer | B-8734 | / | Evonik Industries |
| Modified | Desmodur 3133 | / | Covestro AG |
| isocyanate | | | |

### Example 1

### (1) Preparation of a Phosphazene Catalyst

step 1: 10 g of phosphorus pentachloride and 90 g of propionitrile solution were added into a 500 ml three-necked flask equipped with a stirrer, a thermometer, and a dropping funnel, 46 g of tetramethylguanidine was added dropwise under nitrogen protection, a reaction temperature was controlled around 0° C., and atmospheric pressure was maintained as a reaction pressure; after the addition was completed, the temperature was slowly brought back to room temperature to obtain a mixture, the mixture was stirred for 0.5 h, transferred to an oil bath and stir at 110° C. for 3 h, and then cooled to room temperature and filtered to remove a precipitate, so as to obtain a solution; and

step 2: 3.7 g of potassium methoxide was added to the solution obtained in the step 1 to obtain a mixture, the mixture was reacted at 60° C. and atmospheric pressure for 3 h, then cooled to room temperature and filtered to collect a filtrate, distillation was performed under reduced pressure to obtain 23.6 g of unpurified dark oily phosphazene catalyst with a yield of 94.8%.

(2) Preparation of polyether polyol: 120 g of CHE-307 polyether and 3.0 g of the phosphazene catalyst were added in a 2 L high-pressure reactor, replaced with nitrogen three times, and degassed at 105° C. under-0.09 MPa pressure for 2 h, in which case, a value of n in General Formula (II) was 0.025. 1130 g of propylene oxide was added dropwise for polymerization under a pressure of −0.09 MPa and a temperature of 95° C. in the reactor; nitrogen was charged to stabilize the pressure inside the reactor, and residual monomers were removed under vacuum negative pressure; 250 g of ethylene oxide was added dropwise at a slight positive pressure and a temperature of 105° C. for end-capping, and nitrogen was charged and aged until the pressure inside the reactor remained unchanged; and pure water and magnesium silicate adsorbent were added, and stirred at 105° C. for 1 h, vacuum dehydration was then performed under negative pressure and filtered to obtain the desired polyether polyol.

(3) Preparation of polyurethane foam material: Preparation of Component A: 100 parts of the above polyether polyol, 1 part of diethanolamine, 2 parts of Dabco NE-1091, 1.5 parts of B-8734, and 3.5 parts of water in percent by weight were added in Container A, stirred for 30 min to prepare Component A. Preparation of Component B: 32.5 parts of Desmodur 3133 was added in Container B, and Components A and B were preheated to 50° C.; Components A and B were injected into a mold through a high-pressure foaming machine, or a low-pressure foaming machine; and a mold temperature was set to 50° C., and the mold was opened after 180 s to take the material out of the mold to obtain a low-odor polyurethane foam material.

Performance testing data for the prepared phosphazene catalyst, polyether polyol, and polyurethane foam material were shown in Tables 3-5.

### Example 2

### (1) Preparation of a Phosphazene Catalyst

step 1: 10 g of phosphorus pentachloride and 90 g of propionitrile solution were added into a 500 ml three-necked flask equipped with a stirrer, a thermometer, and a dropping funnel, 46 g of tetramethylguanidine was added dropwise under nitrogen protection, a reaction temperature was controlled around 0° C., and atmospheric pressure was maintained as a reaction pressure; after the addition was completed, the temperature was slowly brought back to room temperature to obtain a mixture, the mixture was stirred for 0.5 h, transferred to an oil bath and stir at 110° C. for 3 h, and then cooled to room temperature and filtered to remove a precipitate, so as to obtain a solution; and

step 2: 3.7 g of potassium methoxide was added to the solution obtained in the step 1 to obtain a mixture, the mixture was reacted at 60° C. and atmospheric pressure for 3 h, then cooled to room temperature and filtered to collect a filtrate, distillation was performed under reduced pressure to obtain a concentrated solution, the concentrated solution was recrystallized using acetonitrile and cyclohexane, where a volume ratio of acetonitrile to cyclohexane (v/v)=1:50, filtering and drying were then performed to obtain 19.4 g of purified white powdery phosphazene catalyst, with a yield of 78%. A structural general formula of the obtained phosphazene catalyst was shown in General Formula (I) above.

(2) Preparation of polyether polyol: 120 g of CHE-307 polyether and 2.25 g of the phosphazene catalyst were added in a 2 L high-pressure reactor, replaced with nitrogen three times, and degassed at 105° C. under −0.09 MPa pressure for 2 h, in which case, a value of n in General Formula (II) was 0.025. 1130 g of propylene oxide was added dropwise for polymerization under a pressure of −0.09 MPa and a temperature of 95° C. in the reactor; nitrogen was charged to stabilize the pressure inside the reactor, and residual monomers were removed under vacuum negative pressure; 250 g of ethylene oxide was added dropwise at a slight positive pressure and a temperature of 105° C. for end-capping, and nitrogen was charged and aged until the pressure inside the reactor remained unchanged; and pure water and magnesium silicate adsorbent were added, and stirred at 105° C. for 1 h, vacuum dehydration was then performed under negative pressure and filtered to obtain the desired polyether polyol.

(3) Preparation of polyurethane foam material: Preparation of Component A: 100 parts of the above polyether polyol, 1 part of diethanolamine, 2 parts of Dabco NE-1091, 1.5 parts of B-8734, and 3.5 parts of water in percent by weight were added in Container A, stirred for 30 min to prepare Component A. Preparation of Component B: 32.5 parts of Desmodur 3133 was added in Container B, and Components A and B were preheated to 50° C.; Components A and B were injected into a mold through a high-pressure foaming machine, or a low-pressure foaming machine; and a mold temperature was set to 50° C., and the mold was opened after 180 s to take the material out of the mold to obtain a low-odor polyurethane foam material.

Performance testing data for the prepared phosphazene catalyst, polyether polyol, and polyurethane foam material were shown in Tables 3-5.

### Examples 3-12

Examples 3-12 were carried out according to the steps of Example 2, except that the General Formulation of phosphazene catalyst is different. Specific General Formulation and relevant conditions were shown in Table 2; and performance test data of the prepared phosphazene catalysts, polyether polyol, and polyurethane foam material are shown in Tables 3-5.

### Comparative Example 1

Comparative Example 1 is a phosphazene catalyst prepared using toluene as the solvent according to Example 1 in Patent No. CN104497046B.

### Comparative Example 2

Comparative Example 2 is a phosphazene catalyst prepared using toluene according to Synthesis Example 1 in Patent No. CN102171272B.

### Comparative Example 3

Comparative Example 3 is a phosphazene catalyst prepared using toluene according to the method in Examples 1, 2, and 5 in Patent No. DE102006010034.

Steps of the phosphazene catalysts obtained in Comparative Examples 1-3 that were used in the preparation of polyether polyols and polyurethane foam materials were the same as those for Examples 1-12, and performance test data for the phosphazene catalysts, polyether polyols, and polyurethane foam materials were shown in Tables 3-5.

TABLE 2
Specific General Formulation and related conditions of Examples 1-12

| Example | Example | Example | Example | Example | Example | Example |
|---|---|---|---|---|---|---|
| 1 | 2 | 3 | 4 | 5 | 6 | 7 |

Step 1

| | | | | | | | |
|---|---|---|---|---|---|---|---|
| Phosphorus | 10 | 10 | 10 | 10 | 10 | 10 | 10 |
| pentachloride (g) | | | | | | | |
| Tetramethylguanidine | 46 | 46 | 45.3 | 44.2 | 49.8 | 55.3 | 38.7 |
| (g) | | | | | | | |
| Propionitrile (g) | 90 | 90 | / | / | / | / | / |
| Nitrile (g) | / | / | 80 | / | / | / | / |
| Adiponitrile (g) | / | / | / | 70 | / | / | / |
| Propyl ether (g) | / | / | / | / | 100 | / | / |
| Butyl ether (g) | / | / | / | / | / | 110 | / |
| 1,4-dioxane (g) | / | / | / | / | / | / | 120 |
| Dropwise | 0 | 0 | 0 | 0 | 0 | 0 | 0 |
| temperature (° C.) | | | | | | | |
| Stirring duration (h) | 0.5 | 0.5 | 0.5 | 0.5 | 0.5 | 0.5 | 0.5 |
| Stirring duration for | 3 | 3 | 4 | 5 | 6 | 7 | 8 |
| oil bath (h) | | | | | | | |
| Temperature for oil | 110 | 110 | 80 | 90 | 100 | 110 | 120 |
| bath (° C.) | | | | | | | |

Step 2

| Potassium methoxide | 3.7 | 3.7 | 4.1 | / | / | / | / |
|---|---|---|---|---|---|---|---|
| (g) | | | | | | | |
| Sodium methoxide | / | / | / | 2.6 | 3.6 | / | / |
| (g) | | | | | | | |
| Potassium acetate (g) | / | / | / | / | / | 6.6 | / |
| Sodium acetate (g) | / | / | / | / | / | / | 6.3 |
| Potassium | / | / | / | / | / | / | / |
| dihydrogen | | | | | | | |
| phosphate (g) | | | | | | | |
| Sodium dihydrogen | / | / | / | / | / | / | / |
| phosphate (g) | | | | | | | |
| Reaction temperature | 60 | 60 | 50 | 60 | 70 | 80 | 80 |
| (° C.) | | | | | | | |
| Reaction time (h) | 3 | 3 | 4 | 5 | 6 | 7 | 8 |
| Volume ratio of | / | Acetonitrile:cyclo- | Acetonitrile:n- | / | / | Methanol:n- | Methanol:cyclo- |
| purification | | hexane = 1:50 | hexane = 1:50 | | | hexane = 1:80 | hexane = 1::00 |
| solvent | | | | | | | |
| (recrystallization) | | | | | | | |
| Volume ratio of | / | / | / | Acetonitrile:n- | Propanenitrile:petro- | / | / |
| purification solvent | | | | heptane = 1:100 | leum ether = 1:140 | | |
| (slurry) | | | | | | | |
| Yield before | 94.8 | 94.8 | 94.2 | 93.3 | 96.3 | 97.2 | 96.9 |
| purification (%) | | | | | | | |
| Yield after | / | 78 | 77 | 72 | 80 | 82 | 79 |
| purification (%) | | | | | | | |
| Catalyst (g) | 3 | 2.25 | | | | | |
| Amount of feeding of | / | / | / | 100 | 80 | 50 | / |
| CHE-204 (g) | | | | | | | |
| Amount of feeding of | 120 | 120 | 60 | / | / | / | / |
| CHE-307 (g) | | | | | | | |
| Amount of feeding of | / | / | / | / | / | / | 30 |
| CHE-628 (g) | | | | | | | |
| Formula (II): value | 0.025 | 0.025 | 0.05 | 0.017 | 0.021 | 0.033 | 0.089 |
| of n | | | | | | | |

| | Example | Example | Example | Example | Example |
|---|---|---|---|---|---|
| | 8 | 9 | 10 | 11 | 12 |
| Step 1 | | | | | |
| Phosphorus | 10 | 10 | 10 | 10 | 10 |
| pentachloride (g) | | | | | |
| Tetramethylguanidine | 46 | 46 | 46 | 46 | 46 |
| (g) | | | | | |
| Propionitrile (g) | 45 | 45 | 90 | 90 | 90 |
| Nitrile (g) | 45 | / | / | / | / |
| Adiponitrile (g) | / | / | / | / | / |
| Propyl ether (g) | / | 45 | / | / | / |
| Butyl ether (g) | / | / | / | / | / |
| 1,4-dioxane (g) | / | / | / | / | / |
| Dropwise | 0 | 0 | 0 | 0 | 0 |
| temperature (° C.) | | | | | |
| Stirring duration (h) | 0.5 | 0.5 | 0.5 | 0.5 | 0.5 |
| Stirring duration for | 3 | 3 | 3 | 3 | 3 |
| oil bath (h) | | | | | |
| Temperature for oil | 110 | 110 | 110 | 110 | 110 |
| bath (° C.) | | | | | |
| Step 2 | | | | | |
| Potassium methoxide | / | / | 3.7 | 3.7 | 3.7 |
| (g) | | | | | |
| Sodium methoxide | / | / | / | / | / |
| (g) | | | | | |
| Potassium acetate (g) | / | / | / | / | / |
| Sodium acetate (g) | / | / | / | / | / |
| Potassium | 10.5 | / | / | / | / |
| dihydrogen | | | | | |
| phosphate (g) | | | | | |
| Sodium dihydrogen | / | 10.4 | / | / | / |
| phosphate (g) | | | | | |
| Reaction temperature | 70 | 60 | 60 | 60 | 60 |
| (° C.) | | | | | |
| Reaction time (h) | 3 | 3 | 3 | 3 | 3 |
| Volume ratio of | / | / | Acetonitrile:cyclo- | Acetonitrile:cyclo- | Acetonitrile:cyclo- |
| purification | | | hexane = 1:50 | hexane = 1:50 | hexane = 1:50 |
| solvent | | | | | |
| (recrystallization) | | | | | |
| Volume ratio of | n-butanol:n- | Methanol:petro- | / | / | / |
| purification solvent | heptane = | leum ether = 1:200 | | | |
| (slurry) | 1:160 | | | | |
| Yield before | 96.1 | 94.3 | 94.8 | 94.8 | 94.8 |
| purification (%) | | | | | |
| Yield after | 77 | 76 | 78 | 78 | 78 |
| purification (%) | | | | | |
| Catalyst (g) | 2.25 | | | | |
| Amount of feeding of | / | / | / | 0.87 | / |
| CHE-204 (g) | | | | | |
| Amount of feeding of | / | / | 3 | / | / |
| CHE-307 (g) | | | | | |
| Amount of feeding of | 10 | 5 | / | / | 0.47 |
| CHE-628 (g) | | | | | |
| Formula (II): value | 0.25 | 0.51 | 1 | 2 | 5.9 |
| of n | | | | | |

TABLE 3
Data results of phosphazene catalysts in Examples 1-12 and Comparative Examples 1-3 (Unit: ug/m³)

| | Example | Example | Example | Example | Example | Example | Example | Example |
|---|---|---|---|---|---|---|---|---|
| | 1 | 2 | 3 | 4 | 5 | 6 | 7 | 8 |
| Odor level | 4 | 3.5 | 3.5 | 3.5 | 3.5 | 3.5 | 3.5 | 3.5 |
| (25° C.) | | | | | | | | |
| Benzopyrene | ND | ND | ND | ND | ND | ND | ND | ND |
| Methylbenzene | ND | ND | ND | ND | ND | ND | ND | ND |
| Ethylbenzene | ND | ND | ND | ND | ND | ND | ND | ND |
| Xylene | ND | ND | ND | ND | ND | ND | ND | ND |
| (o-Xylene, | | | | | | | | |
| m-Xylene, | | | | | | | | |
| p-Xylene) | | | | | | | | |
| TVOC | 329.7 | 162.6 | 170.3 | 166.9 | 172.7 | 155.2 | 160.5 | 161.1 |
| (C6-C16) | | | | | | | | |
| Yield after | / | 78 | 77 | 72 | 80 | 82 | 79 | 77 |
| purification | | | | | | | | |
| (%) | | | | | | | | |

| | | | | | Comparative | Comparative | Comparative |
|---|---|---|---|---|---|---|---|
| | Example | Example | Example | Example | Example | Example | Example |
| | 9 | 10 | 11 | 12 | 1 | 2 | 3 |
| Odor level | 3.5 | 3.5 | 3.5 | 3.5 | 5.5 | 4.0+ | 4.0+ |
| (25° C.) | | | | | | | |
| Benzopyrene | ND | ND | ND | ND | ND | ND | ND |
| Methylbenzene | ND | ND | ND | ND | 3445.4 | ND | 198.371 |
| Ethylbenzene | ND | ND | ND | ND | ND | ND | ND |
| Xylene | ND | ND | ND | ND | 14.5 | 19.9 | 40.9 |
| (o-Xylene, | | | | | | | |
| m-Xylene, | | | | | | | |
| p-Xylene) | | | | | | | |
| TVOC | 1579 | 162.6 | 162.6 | 162.6 | 148772.1 | 252.3 | 249.3 |
| (C6-C16) | | | | | | | |
| Yield after | 76 | 78 | 78 | 78 | / | 81 | 77 |
| purification | | | | | | | |
| (%) | | | | | | | |

TABLE 4
Data results of polyether polyols prepared with catalysts
in Examples 1-12 and Comparative Examples 1-3 (Unit: ug/m³)

| | Example | Example | Example | Example | Example | Example | Example | Example |
|---|---|---|---|---|---|---|---|---|
| | 1 | 2 | 3 | 4 | 5 | 6 | 7 | 8 |
| Odor level | 4.0+ | 3.5− | 3.5− | 3.5− | 3.5− | 3.5− | 3.5− | 3.5− |
| (80° C.) | | | | | | | | |
| Benzopyrene | ND | ND | ND | ND | ND | ND | ND | ND |
| Methylbenzene | ND | ND | ND | ND | ND | ND | ND | ND |
| Ethylbenzene | ND | ND | ND | ND | ND | ND | ND | ND |
| Xylene | ND | ND | ND | ND | ND | ND | ND | ND |
| (o-Xylene, | | | | | | | | |
| m-Xylene, | | | | | | | | |
| p-Xylene) | | | | | | | | |
| TVOC | 302.4 | 200.3 | 211.9 | 210.8 | 205 | 202.1 | 211.7 | 209.7 |
| (C6-C16) | | | | | | | | |
| Formaldehyde | 50 | 35.5 | 39.7 | 37.7 | 31.1 | 31.7 | 33.9 | 35.8 |
| Acetaldehyde | 144.6 | 25.3 | 23.9 | 25.8 | 21.1 | 21.3 | 29 | 29.1 |
| Propionaldehyde | 19.9 | ND | ND | ND | ND | ND | ND | ND |
| Acrolein | 23.3 | ND | ND | ND | ND | ND | ND | ND |

| | | | | | Comparative | Comparative | Comparative |
|---|---|---|---|---|---|---|---|
| | Example | Example | Example | Example | Example | Example | Example |
| | 9 | 10 | 11 | 12 | 1 | 2 | 3 |
| Odor level | 3.5− | 3.5− | 3.5− | 3.5− | 4.5 | 4.0− | 4.0− |
| (80° C.) | | | | | | | |
| Benzopyrene | ND | ND | ND | ND | 14.9 | ND | ND |
| Methylbenzene | ND | ND | ND | ND | 15.9 | 16 | 18.7 |
| Ethylbenzene | ND | ND | ND | ND | 14.6 | ND | ND |
| Xylene | ND | ND | ND | ND | 18.9 | ND | ND |
| (o-Xylene, | | | | | | | |
| m-Xylene, | | | | | | | |
| p-Xylene) | | | | | | | |
| TVOC | 206.3 | 210.3 | 222.9 | 225.6 | 470.3 | 251.5 | 219.1 |
| (C6-C16) | | | | | | | |
| Formaldehyde | 35.9 | 36.5 | 37.1 | 37.4 | 51.2 | 88.8 | 76.3 |
| Acetaldehyde | 29.3 | 25.7 | 25.9 | 21.4 | 91.1 | 625.7 | 303.6 |
| Propionaldehyde | ND | ND | ND | ND | 33.5 | 88.8 | 33.1 |
| Acrolein | ND | ND | ND | ND | 25.9 | ND | 20.3 |

TABLE 5
Data results of polyurethane foam materials prepared with polyols
in Examples 1-12 and Comparative Examples 1-3 (Unit: ug/m³)

| | Example | Example | Example | Example | Example | Example | Example | Example |
|---|---|---|---|---|---|---|---|---|
| | 1 | 2 | 3 | 4 | 5 | 6 | 7 | 8 |
| Odor level | 4.0+ | 3.5− | 3.5− | 3.5− | 3.5− | 3.5− | 3.5− | 3.5− |
| (80° C.) | | | | | | | | |
| Benzopyrene | ND | ND | ND | ND | ND | ND | ND | ND |
| Methylbenzene | 33.5 | 31.2 | 39.2 | 40 | 48.1 | 40.2 | 31.6 | 31 |
| Ethylbenzene | 14.4 | 13.6 | 13.7 | 13.6 | 13.8 | 13.6 | 12.7 | 12.2 |
| Xylene | 43.7 | 48.9 | 41.6 | 44.1 | 47.8 | 41.9 | 47.6 | 48.9 |
| (o-Xylene, | | | | | | | | |
| m-Xylene, | | | | | | | | |
| p-Xylene) | | | | | | | | |
| Styrene | ND | ND | ND | ND | ND | ND | ND | ND |
| TVOC | 3203.5 | 1076.9 | 1113.4 | 1326.1 | 1216.9 | 978 | 1019.1 | 1116.7 |
| (C6-C16) | | | | | | | | |
| Formaldehyde | 190.8 | 98.1 | 90.2 | 94.2 | 98.9 | 91.9 | 92.3 | 98.6 |
| Acetaldehyde | 52.4 | 43.4 | 40 | 44.2 | 44.2 | 42.2 | 43.1 | 41.1 |
| Propionaldehyde | 77.8 | 47.9 | 45.7 | 46.2 | 41.3 | 41.9 | 45.9 | 46.2 |

| | | | | | Comparative | Comparative | Comparative |
|---|---|---|---|---|---|---|---|
| | Example | Example | Example | Example | Example | Example | Example |
| | 9 | 10 | 11 | 12 | 1 | 2 | 3 |
| Odor level | 3.5− | 3.5− | 3.5− | 3.5− | 4.5+ | 4 | 4 |
| (80° C.) | | | | | | | |
| Benzopyrene | ND | ND | ND | ND | 12.7 | ND | ND |
| Methylbenzene | 38.3 | 33.1 | 32.8 | 31.5 | 170.4 | 54.8 | 88.4 |
| Ethylbenzene | 12.1 | 13 | 12.6 | 13.2 | 14.7 | 14.9 | 15.6 |
| Xylene | 47.7 | 44.2 | 47.9 | 45.1 | 40.4 | 62.7 | 72.6 |
| (o-Xylene, | | | | | | | |
| m-Xylene, | | | | | | | |
| p-Xylene) | | | | | | | |
| Styrene | ND | ND | ND | ND | ND | ND | ND |
| TVOC | 1134.5 | 1101.2 | 1195.6 | 1043.3 | 7971.4 | 2649.8 | 2930.4 |
| (C6-C16) | | | | | | | |
| Formaldehyde | 90.5 | 92.3 | 95.1 | 90.5 | 209.5 | 445.6 | 401.2 |
| Acetaldehyde | 40.1 | 43.5 | 41.9 | 42.6 | 16.9 | 41.5 | 42.3 |
| Propionaldehyde | 46.1 | 45.3 | 47.8 | 46.6 | 20.7 | 18.8 | 45.6 |

Note: The odor level in the table above was measured according to the VDA270 standard of the automotive industry. Data of foam were measured at 65° C. using a 10 L sampling bag method: a detection limit for benzene series was 11.342 ug/m³, and a detection limit for aldehydes and ketones was 18.903 ug/m³. Data of catalysts, polyether polyols, and other raw materials were measured at 80° C. using the 10 L sampling bag method: a detection limit for benzene series was 11.845 ug/m³, and a detection limit for aldehydes and ketones was 19.742 ug/m³.

What is claimed is:

1. A continuous preparation method for a low-odor phosphazene catalyst, comprising the following steps:
(1) dissolving phosphorus pentahalide in an organic solvent to obtain an organic phosphorus pentahalide solution;
adding dropwise a solution of guanidine compound to the organic phosphorus pentahalide solution under an inert atmosphere and at a temperature of −15° C.-5° C.; and
bringing a temperature of the solution to room temperature after the addition is completed, stirring at the room temperature, then heating in an oil bath to facilitate continuous reaction, cooling to the room temperature upon completion of the reaction, filtering to remove a precipitate to obtain an organic phosphonium salt solution containing the organic solvent and a halogenated phosphorus compound; and
(2) adding an alkali metal salt compound to the organic phosphonium salt solution obtained in the step (1); and
performing the reaction under the inert atmosphere and heating conditions, cooling and filtering after the completion of the reaction, and distilling the filtrate under a reduced pressure to obtain a crude product of phosphazene compound,
wherein the organic solvent is selected from at least one of nitriles or ethers,
the alkali metal salt compound is selected from one of sodium alkoxide, potassium alkoxide, sodium carboxylate, potassium carboxylate, or phosphate metal salt, and
the low-odor phosphazene catalyst comprises a phosphazene cation and an alkali metal salt compound anion represented by General Formula (I), 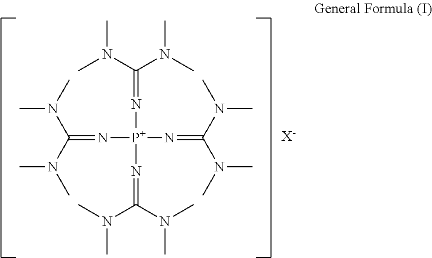
in the above General Formula (I), X represents the alkali metal salt compound, and X-represents an alkali metal anion formed by a departure of a metal ion from X.

2. The continuous preparation method for a low-odor phosphazene catalyst according to claim 1, wherein the organic solvent is selected from at least one of propionitrile, butyronitrile, adiponitrile, propyl ether, butyl ether, or 1,4-dioxane, and
the alkali metal salt compound is selected from one of potassium methoxide, sodium methoxide, potassium ethoxide, sodium ethoxide, potassium formate, sodium formate, potassium acetate, sodium acetate, potassium dihydrogen phosphate, or sodium dihydrogen phosphate.

3. The continuous preparation method for a low-odor phosphazene catalyst according to claim 1, wherein in the step (1), a mass ratio of the phosphorus pentahalide to the organic solvent is 1:(6-12),
a molar ratio of the phosphorus pentahalide to the guanidine compound is 1:(5-11),
the temperature of the addition is 0-5° C.,
a temperature of heating in the oil bath is 80-120° C., and
a stirring reaction of heating in the oil bath lasts for 3-8 h.

4. The continuous preparation method for a low-odor phosphazene catalyst according to claim 1, wherein in the step (2), a molar ratio of the organic phosphonium salt solution to the alkali metal salt compound is 1:(1-2),
a reaction temperature of the heating conditions is 50-80° C., and
the reaction under the heating conditions lasts for 3-8 h.

5. The continuous preparation method for a low-odor phosphazene catalyst according to claim 1, wherein in the step (1), the phosphorus pentahalide is selected from phosphorus pentabromide or phosphorus pentachloride, and
the guanidine compound is 1,1,3,3-tetramethylguanidine.

6. The continuous preparation method for a low-odor phosphazene catalyst according to claim 1, wherein the crude product of phosphazene compound obtained in the step (2) is subjected to recrystallization or slurry for purification,
steps for the recrystallization are as follows: the crude product of phosphazene compound is subjected to recrystallization using a purification solvent, then filtering and drying to obtain a white powdery low-odor phosphazene catalyst, wherein the purification solvent is selected from a mixed solution of at least one of nitriles or alcohols and at least one of ethers or alkanes, and a volume ratio of the at least one of nitriles or alcohols to the at least one of ethers or alkanes is 1:(2-300), and
steps for the slurry are as follows: the crude product of phosphazene compound is subjected to slurry using a purification solvent, then filtering and drying to obtain the white powdery low-odor phosphazene catalyst, wherein the purification solvent is selected from a mixed solution of at least one of nitriles or alcohols and at least one of ethers or alkanes, and a volume ratio of the at least one of nitriles or alcohols to the at least one of ethers or alkanes is 1:(2-500).

7. A method of using a low-odor phosphazene catalyst prepared by the method according to claim 1 in a preparation of polyether polyols and polyurethane foam materials.

8. The method according to claim 7, wherein the low-odor phosphazene catalyst represented in the General Formula (I) is mixed with an active hydrogen compound Y and subjected to heating treatment to obtain a salt of phosphazene cation and active hydrogen compound anion represented in General Formula (II), 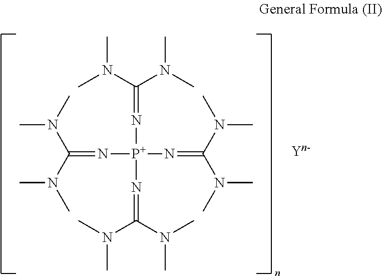
in the above General Formula (II), n is a real number greater than 0 but less than or equal to 8, and Yⁿ-represents an anion of the active hydrogen compound formed by a departure of n protons from the active hydrogen compound Y, and
Y is a polyether polyol with a functionality of 2-8 and a molecular weight of 300-2000.

9. The method according to claim 8, wherein n is a real number greater than 0 but less than or equal to 1, and
Y is a polyether polyol with a functionality of 2-6 and a molecular weight of 400-1200.
